(19) United States
(12) Patent Application Publication
(10) Pub. No.: US 2004/0043508 A1
(43) Pub. Date: Mar. 4, 2004

(54) Polymer-coated substrates for binding biological molecules

(76) Inventors: Anthony G. Frutos, Painted Post, NY (US); Joydeep Lahiri, Painted Post, NY (US); Thomas M. Leslie, Horseheads, NY (US); Jinlin Peng, Painted Post, NY (US); Dana C. Bookbinder, Corning, NY (US); Xinying Xie, Painted Post, NY (US)

Correspondence Address: CORNING INCORPORATED, SP-TI-3-1, CORNING, NY, 14831

(21) Appl. No.: 10/234,412

(22) Filed: Sep. 3, 2002

(51) Int. Cl. C12Q 1/68; C12M 1/34; G01N 33/543

(52) U.S. Cl. 436/518; 435/287.2; 435/6

(57) ABSTRACT

A substrate, which that is capable of attaching biomolecules, and a method for preparing the substrate are provided. The substrate has a reactive surface that can covalently attach a polymer coating containing functional groups, which can reduce nonspecific binding of biomolecules to the surface for a biological array. Optionally, at least a portion of the substrate may be coated with an intermediate tie layer, which enhances the covalent bonding between the polymer coating with the underlying substrate. The present invention also pertains to a method that uses electrostatic blocking agents to reduce non-specific binding of proteins to a substrate, especially anhydride-modified surfaces.

### FIELD OF INVENTION

The present invention relates to an improved substrate onto which arrays of biological molecules may be immobilized, and to the biological arrays incorporating the improved substrate. The present invention further relates to methods for preparing the substrate and inhibiting nonspecific binding to the arrays.

### BACKGROUND OF THE INVENTION

Biological arrays have been used for high-throughput assays in various biological, clinical, or pharmaceutical studies. Arrays may contain a chosen collection of biological molecules (a.k.a., biomolecules), such as probes specific for important pathogens, genetic sequence markers, antibodies, immunoglobulins, receptor proteins, peptides, cells, and the like. For instance, an array can have a collection of oligonucleotides specific for known sequence markers of genetic diseases, or probes to isolate a desired protein from a biological sample. A biological array may comprise a number of different, individual biomolecules tethered to the surface of a substrate in a regular pattern, each one in a distinct spot, so that the location of each biomolecule is known.

Biomolecule arrays can be synthesized on a substrate according to an assortment of methods. For example, to produce an array directly on a substrate, one may employ methods of solid-phase chemical synthesis in combination with site-directing mass as disclosed in U.S. Pat. No. 5,510,270, incorporated herein by reference. Alternatively, one may use photolithographic techniques involving precise drop deposition via piezoelectric pumps, as disclosed in U.S. Pat. No. 5,474,796, incorporated herein by reference. Or, one may contact a substrate with typographic pins holding droplets and using ink jet printing mechanisms to lay down an array matrix.

Examples of commercially available substrates for immobilization of biomolecules include products such as SuperAldehyde™ from CloneTech or 3D link™ slides from Motorola, formerly Surmodics. The SuperAldehyde™ slide requires an additional reduction step to stabilize a covalent attachment between the slide and the biomolecule. This feature causes problems in some heterogeneous assays since the reduction step may damage biomolecules attached to the surface, thus reducing their effectiveness in an assay. The Motorola slides, on the other hand, suffer from a relatively slow reaction-kinetic rate, requiring longer reaction times, typically over 6 or 12 hours, for biomolecules to attach to the surface in sufficient amounts. Although some researchers have tried to develop a functionalizable polymer interlayer or cushion, which reduces non-specific binding of cells (e.g., D. Beyer et al., Langmuir 1996, 12, 2514-2518; Langmuir 1998, 14, 3030-3035, incorporated herein by reference), they have not been able to shorten the relatively long reaction time for attaching biological analytes.

In view of the shortcomings and limitations of currently available devices, a need exists for an improved substrate that reduces nonspecific binding of biological molecules as well as an alternative surface chemistry for faster binding kinetics.

### SUMMARY OF THE INVENTION

The present invention pertains, in part, to a substrate that has a reactive surface to which a polymer coating can attach by covalent bonds. The invention also relates to a method of preparing such a substrate for a biological assay device. The substrate has an even coating of polymer or copolymers containing functional groups, which can reduce nonspecific binding of biomolecules to the polymer-coated surface for a biological array. In other words, functional groups or charges on the polymer coating that interact with groups or charges on the biomolecules to attach or immobilize the biomolecules to the polymer coating. The present invention also pertains to a biological array formed by the attachment of biomolecules on to the substrate according to the method. Biomolecules can attach to the polymer-coated substrate in sufficient amounts to form microspots within about 5 or 5.5 hours, typically about 4 or 4.5 hours, and preferably within about 2 or 3.5 hours.

According to the present invention, the method for preparing the polymer-coated substrate includes several steps: providing a substrate; preparing a reactive surface on the substrate for attaching a polymer coating; and, applying the polymer coating to the reactive surface of the substrate. Other steps may include subsequently treating the surface with other chemical reagents to create a stable attachment having a reduced background signal, and depositing biomolecules onto the polymer-coated surface.

Depending on the nature of the underlying substrate, an intermediate tie layer containing functional groups may be used to enhance covalent bonds between the substrate and the polymer coating. In other words, when the substrate is absent a surface capable of chemically engaging or attaching the polymer coating, depositing a tie layer having appropriate functional groups will be necessary to prepare the reactive surface of the substrate. Such functional groups may include an amino group, thiol group, hydroxyl group, carboxyl group, organic and inorganic acid, and their derivatives or salts.

When the functional groups on the polymer coating react with the underlying substrate, they may form a uniform negative charge on the substrate, which is potentially useful in decreasing background signals for nucleic acid hybridization applications in a heterogeneous assay. The polymer coating may include anhydrides, and preferably, is not soluble in water. In accordance with the present invention, the polymer coating can be as thin as a monolayer; however, preferably is slightly thicker to provide a uniform, even coating over the substrate surface. For instance, the polymer layer may be as thin as about 20 Å or 25 Å. More preferably, the polymer coating has a thickness in the range of about 50-1000 Å or greater. In further embodiments, the polymer coating contains a copolymer having a combination of, for example, but not limited to, maleic anhydride and styrene, divinylbenzene, tetradecene, octadecene or butylvinyl ether.

Various kinds of biological moieties may be immobilized according to the present invention. Not to be limiting, some biomolecules may include, for example, probes specific for pathogens, sequence markers, antibodies, immunoglobulins, proteins, peptides, nucleic acids, oligonucleotides, cells, and the like. The biomolecules are attached to the polymer coating by covalent binding, electrostatic interactions or a combination thereof.

The polymer coating can be used with a variety of underlying substrates, which may be of gold, silver, platinum, plastic, polymer, ceramic, chromium, or glass materials, where glass is preferred. Using the substrate of the present invention, biological arrays of, for example, short oligonucleotides can be formed.

In another aspect, the present invention relates to a novel blocking method, which is based on electrostatic binding of charged compounds to a surface of an opposite charge, such as positively charged compounds on surfaces modified with anhydride-containing polymers. The procedure should make polymeric anhydride-modified surfaces useful for the study of protein-protein and protein-ligand interactions. Contacting the polymer-coated surface, for example, with a positively charged dextran layer (e.g., diethylaminoethyl (DEAE) dextran) can reduce significantly the amount of non-specific protein binding to a negatively charged array surface, as compared to more traditional blocking agents.

Additional features and advantages of the present method and array device will be disclosed in the following detailed description. It is understood that both the foregoing general description and the following detailed description and examples are merely representative of the invention, and are intended to provide an overview for understanding the invention as claimed.

### BRIEF DESCRIPTION OF THE DRAWINGS

FIG. 1A shows an embodiment of the present invention, in which a polymer coating reacts to form covalent bonds with both a substrate and a functional group on a biomolecule.

FIG. 1B shows an alternate embodiment of the covalent attachment of a number of biomolecules to a functional polymer layer of a thickness greater than a monolayer, which in turn is linked covalently to a polar moiety attached to the surface of the substrate. Individual units within the polymer layer may be cross-linked with each other.

FIG. 2 shows the effect that pH and copolymer anhydride content in the polymer coating on a substrate has on the attachment of primary amine-modified oligonucleotides labeled with a kind of fluorophore, as measured in RFU. For both low and high anhydride concentrations, 27% SMA and 14% SMA, pH levels of about 9 or higher show enhanced attachment.

FIG. 3 shows a comparison of hybridization signals between a 27% SMA-coated surface and a 14% SMA-coated surface using a pair of complementary 24 mer oligonucleotides, one of which is labeled with Cy5. The x-axis refers to the concentration of printed oligonucleotide on the substrate surface; while, the y-axis refers to the relative fluorescence signal of the complementary labeled oligonucleotide after hybridization. With increasing concentration in the six example, one observes a direct correlation impact on oligonucleotide attachment and the fluorescence intensity of the hybridization signal.

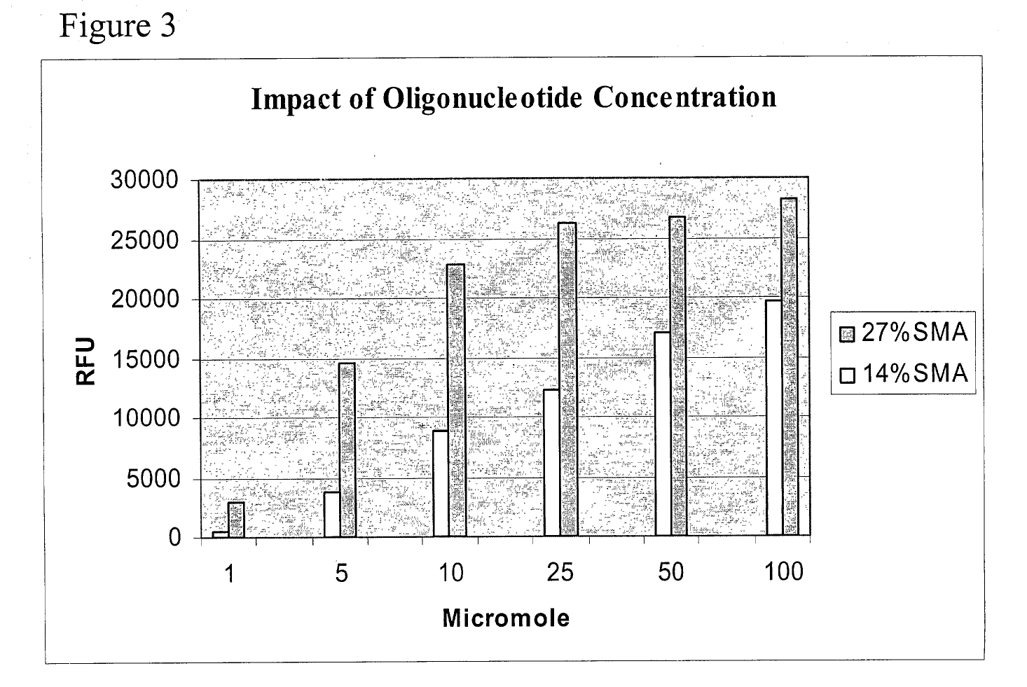

FIGS. 4A-4F show the improvement in both the immobilization by piezo-electric printing of a Cy3 labeled 18 mer oligonucleotide with a borate buffer (pH 9.2/20% DMF) and hybridization of a Cy5 labeled complementary 18 mer oligo due to a higher anhydride content in the SMA slide. FIGS. 4A/4B pertain to an 8% SMA slide; FIGS. 4C/4D pertain to a 14% SMA slide; and FIGS. 4E/4F pertain to a 29% SMA slide.

FIG. 5 shows the preparation of maleic anhydride presenting gold substrates.

FIG. 6 is an SPR sensorgram showing the immobilization of human IgG to a gold surface presenting maleic anhydride groups.

FIG. 7 is an SPR sensorgram showing the specific binding of anti-human IgG to immobilized human IgG.

FIGS. 8A and 8B show results of sensorgrams comparing the binding of proteins to ligands immobilized on (A) maleic anhydride-alt-methyl vinyl ether (see structure 2 in FIG. 5) and (B) styrene maleic anhydride (see structure 1 in FIG. 5).

FIG. 9 shows an SPR experiment examining the non-specific binding of proteins to maleic anhydride modified gold surfaces blocked with ethanolamine (EA) and various kinds of dextrans. Only the surface blocked with DEAE-dextran shows significant increased resistance to the binding of proteins.

FIG. 10 shows an SPR experiment comparing the binding of anti-IgG to surfaces with immobilized IgG that were blocked with either ethanolamine or DEAE dextran. Notice that DEAE dextran does not interfere with anti-IgG binding.

### DETAILED DESCRIPTION

In one aspect the present invention relates, in part, to a substrate that exhibits specific binding characteristics for attaching biological moieties. In another aspect, the present invention relates to a method of forming the substrate used to support an array of biomolecules. According to the invention, the method includes: providing a substrate of a suitable material; preparing on the substrate a reactive surface, which can form covalent bonds with a polymer coating; and, applying the polymer-coating in an even or uniform layer over at least a major surface of the substrate. To create an array, solutions containing biomolecules are deposited at discrete sites on the surface, preferably in a rectilinear matrix having columns and rows. The polymer coating binds a functional group in either the biomolecule or a modified moiety attached to the biomolecule with specificity to at least a part of the coated substrate surface. On a single substrate, one may deposit a plurality of different arrays, as user requirements may dictate. The concentration of the polymer coating in a solvent is in a range of about 0.1-10 wt %/volume. Preferably, the polymer concentration is about 0.5-8% or 1-6%.

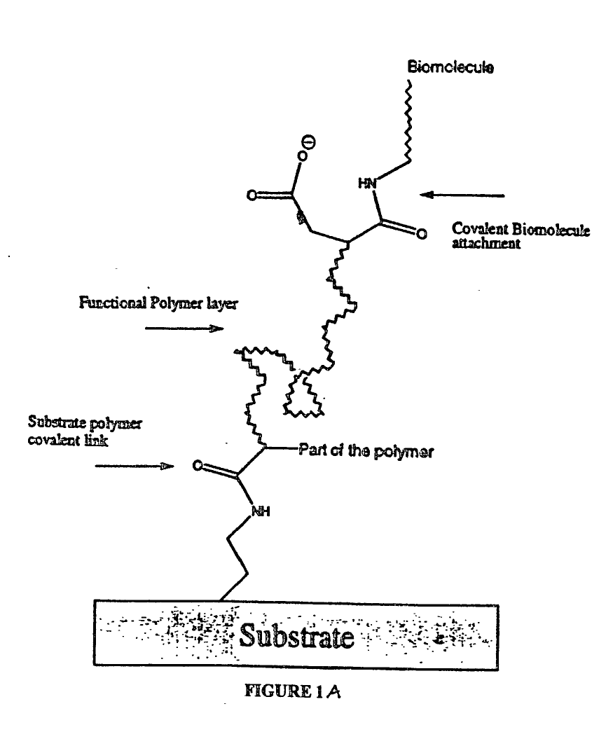

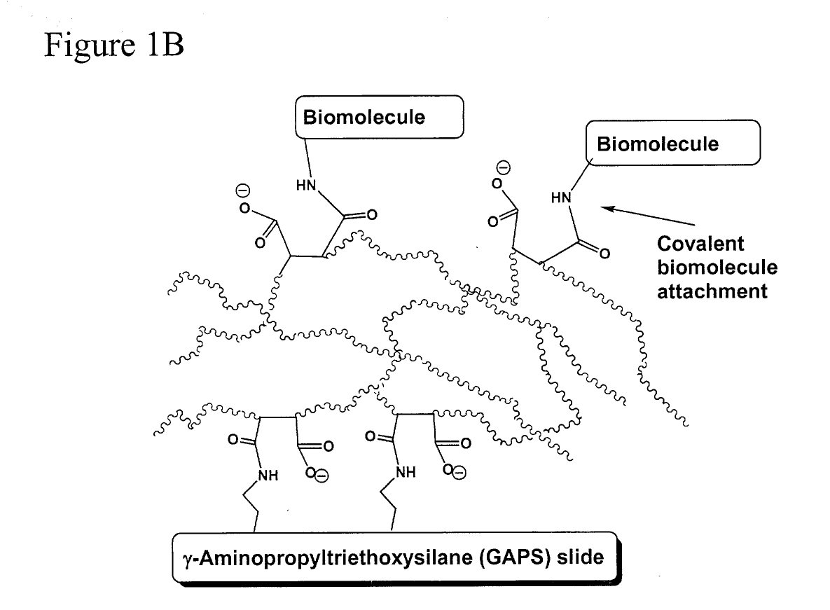

We have found that a coating of a polymer or co-polymers having specific attachment chemistry can create stable substrates for supporting a biological array with reduced or minimal background or nonspecific binding of biomolecules. Moreover, biomolecules can attach to the polymer-coated surface at relatively fast kinetic reaction rates of under about 6 hours, preferably within 5 hours, in amounts to form spots. According to the invention, the polymer coating comprises polymers, copolymers, or other polymeric materials, which have functional groups that can attach by covalent bonds the polymer coating to an underlying substrate, as well as various biological molecules to the polymer-coated substrate surface. FIGS. 1A and 1B depict schematics of two embodiments, in which biomolecules are covalently attached to a functional polymer layer, which in turn is covalently linked to a polar moiety attached to the surface of the substrate. Examples of such polymer functional groups include anhydrides, maleimide, sulfonic acid, acid halide, carboxylic acid, their derivatives or salts.

It is believed that the polymer functional groups react with the substrate surface chemistry to produce residue groups, which create a uniform charge on the substrate at a desired pH level. For instance, according to the present invention, the polymer coating preferably contains an anhydride functional group. A particular advantage of having a polymer coating with anhydride groups is that once the anhydride groups react with the biomolecules and have been exposed to multiple washings, they convert to acid groups in aqueous buffer. Although not intended to be bound by theory, it is believed that these acid groups on the polymer coating produce a uniform negative charge on the coating surface (except at very acidic pH levels of less than about 2.0). This phenomenon in turn helps prevent non-specific binding of nucleic acid to the polymer coating, since both the nucleic acid and surface have negative change and repel each other. The decrease in non-specific binding to the polymer coating reduces background in a heterogeneous assay.

The polymer and copolymers could be linear or non-linear, for example, dendritic polymers. Examples of applicable polymer or copolymers, may include: poly(divinylbenzene), poly(methyl methacrylate), poly(vinyl acetate-maleic anhydride), poly(dimethylsiloxane) monomethacrylate; copolymers such as poly(styrene-co-maleic anhydride), poly(styrene-co-butadiene), poly(styrene-co-divinylbenzene), poly(ethylene-alt-maleic anhydride), poly(isobutylene-alt-maleic anhydride), poly(maleic anhydride-alt-1-octadecene), poly(maleic anhydride-alt-1-tetradecene), poly(2-vinylpyridine-co-styrene), poly(styrene-co-vinylbenzyl chloride-co-divinylbenzene), poly(styrene-co-vinylbenzylamine-co-divinylbenzene), poly(maleic anhydride-alt-methyl vinyl ether), or the like.

In certain embodiments, the polymer coating contains a copolymer of maleic anhydride and another copolymer unit. A copolymer unit may comprise both hydrophilic and hydrophobic units, for example, but not limited to, styrene, divinylbenzene, tetradecene, octadecene, methyl vinyl ether, triethylene glycol methyl vinyl ether or butylvinyl ether. For instance, the polymer coating may be composed of a styrene copolymer, and contain from about 7% to about 50% maleic anhydride, preferably from about 10% to about 33% maleic anhydride, and more preferably from about 14% to about 27-30% maleic anhydride. To avoid de-lamination of the coating from the substrate, preferably, the polymer coating is not soluble in water.

According to the invention, the polymer coating can be as thin as a monolayer, however, preferably is slightly thicker to provide an even coating over the substrate surface. For instance, the polymer layer may be as thin as about 20 Å or 25 Å. More preferably, the polymer coating has a thickness in the range of about 50-1000 Å. In certain embodiments, the polymer coating can be up to a few centimeters thick (e.g., 1-2 or 3 cm).

An assortment of substrates may be employed according to the present invention. The substrate may include any stable solid of a desired dimension selected from either a plastic, a polymer or co-polymer substance, a ceramic, a glass, a metal, a crystalline material, or any combinations thereof, or a coating of one material on another. For example, the substrate can be of (semi) noble metals such as gold or silver; glass materials such as soda glass, quartz glass, Pyrex™ glass, or Vycor™ glass; metallic or non-metallic oxides; silicon, monoammonium phosphate, and other such crystalline materials; transition metals; plastics, polymers or copolymers including dendritic polymers. Preferably, the substrate is planar, in the form of a slide, and is made from a borosilicate or boroaluminosilicate glass. For instance, U.S. Pat. No. 5,374,595, incorporated herein by reference, discloses several glass compositions suitable for use as a substrate in the present invention.

In an alternate embodiment, a rigid, planar substrate or slide can be molded or otherwise made from an anhydride-containing polymer. Such an embodiment would not need another underlying substrate, since the entire substrate could be made of the polymer coating.

Depending on the chemical nature of the underlying substrate, the substrate may be further modified to enable attachment of a polymer coating, a tie layer, or a coating of metallic compositions (e.g., silane, chromium, gold or silver). The functional groups of the polymer coating will bind to either a bare substrate surface or an intermediate tie layer, sandwiched between the polymer coating and underlying substrate. In situations when the surface chemistry of the substrate is less than compatible with the polymer coating, the tie layer can prepare the substrate by providing an intermediate with enhanced attachment chemistry for covalent bonds between the substrate and the polymer coating.

The tie-layer may comprise a variety of reactive polar moieties. Examples of reactive polar moieties may include: amino, hydroxyl, or alkyl-thiol groups, acrylic acid, esters, anhydrides, aldehyde, epoxide or other protected precursors capable of generating reactive functional groups. Reactive polar silane moieties may be straight or branched-chain aminosilane, aminoalkoxysilane, aminoalkylsilane, aminoarylsilane, aminoaryloxysilane, derivatives or salts thereof. Some examples of aminoalkylsilane moieties, which work well in a tie layer, may include: γ-aminopropyl trimethoxysilane, N-(beta-aminoethyl)-γ-aminopropyl trimethoxysilane, N-(beta-aminoethyl)-γ-aminopropyl triethoxysilane or N′-(beta-aminoethyl)-γ-aminopropyl methoxysilane. A preferred example of a polar γ-aminopropylsilane (GAPS) moiety is gamma-aminopropyl trimethoxysilane on a glass surface (available commercially as Corning GAPS™ slides). The tie layer is attached to the substrate by strong chemical interactions, such as by covalent binding. In an alternative embodiment, the tie layer comprises a self-assembled monolayer (SAM). Preferably, when the substrate surface comprises gold, the SAM comprises 11-mercaptoundecylamine or other amine-terminated alkanethiols.

In a preferred embodiment, the underlying substrate has at least a portion coated with a tie-layer. Over the tie layer, the polymer coating is applied and bound to the tie-layer. Biomolecules for an array are immobilized on the polymer coating, which may attach biomolecules by chemical interactions, electrostatic interactions, or combinations thereof.

According to the invention, one may attach several kinds of biomolecules to create assorted biological arrays. The biomolecules may exhibit specific affinity for another molecule through covalent or non-covalent bonding. The biomolecules may include, for example: natural or synthetic oligonucleotides; natural or modified/blocked nucleotides/nucleosides; nucleic acids such as deoxyribonucleic acids (DNA) or ribonucleic acids (RNA); proteins or fragments of proteins; peptides which may contain natural or modified/blocked amino acids; antibodies; haptens; biological ligands; protein or lipid membranes and other biological membranes; cells, etc.

Generally, according to an embodiment, short-length oligonucleotides having about 5-200 base pairs, or preferably 5-100 base pairs that are primary amine-modified, can attach well to the polymer-coated surface. This however, does not necessarily exclude oligonucleotides of longer lengths, such as from about 100 to about 500 bps.

In protein arrays, following covalent attachment of a protein/ligand to a surface, blocking of residual reactive groups on the surface is an important step in the study of protein-protein and/or ligand receptor interactions. Inadequate blocking can lead to high levels of non-specific binding of proteins to the surface, making analysis of results difficult. For example, surfaces based on active-ester (e.g., N-hydroxy succinimide esters) are commonly blocked using ethanolamine to form an amide bond, thereby creating an electrically neutral, hydrophilic surface. In contrast, reaction of an anhydride group with an amine proceeds by a ring-opening mechanism in which both an amide bond and a carboxylic acid are formed, yielding a negatively charged surface (at pH>6). As a consequence, blocking with ethanolamine (EA) or similar reagents is insufficient to block protein as well as DNA. Thus, in another aspect of the present invention, we have developed a method for reducing non-specific binding of proteins to a substrate surface—particularly anhydride-modified surfaces—using electrostatic blocking agents.

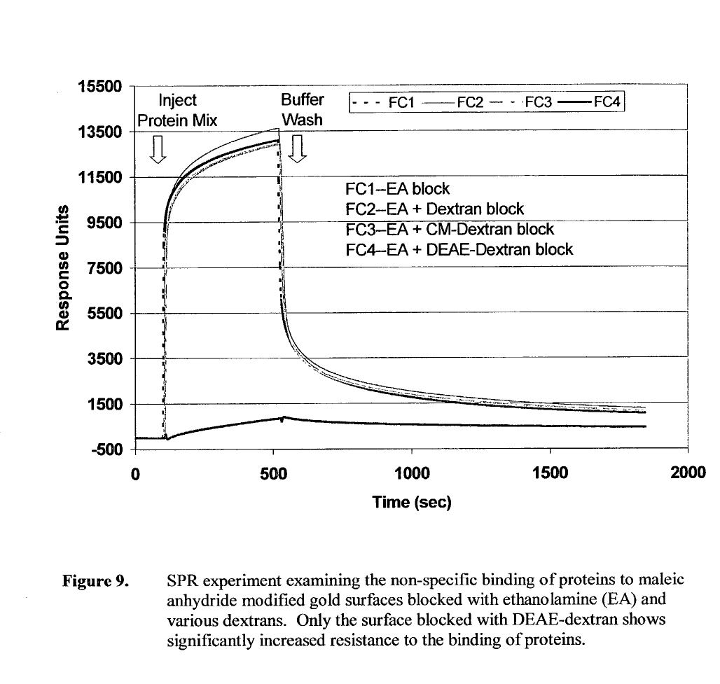

The blocking method comprises contacting the surface with a charged polymer or compound that has good non-specific binding properties itself, after attachment of the biomolecule to the substrate but before a detection step, such as, contacting the array with a target moiety. The charged compound negates a substrate surface of an opposite charge. In other words, it cancels or masks the influence of the substrate. For instance, a compound such as dextran (e.g. DEAE dextran), when with a positive charge, is particularly effective in reducing non-specific binding of proteins to a negatively charged, anhydride-modified surface as compared with more traditional blocking agents such as ethanolamine. (See FIG. 9.)

The examples in the following section further illustrate and describe the advantages and qualities of the present invention.

### EXAMPLES

### Example 1

A. Preparation of Poly[Styrene-co-Maleic Anhydride] (SMA) Coated Slides

Glass slides coated with γ-aminopropyl trimethoxy silane (GAPS), were spin coated with a 5% wt/v poly[styrene-co-maleic anhydride] in dry toluene at about 2000 RPS for about 20 seconds. The slides were dried in a vacuum oven at 100° C. for 1 hour. The slides were then kept in a desiccator until needed. The polymer was also coated onto cleaned plain glass slides for comparison.

B. Attachment of Primary-Amine-Modified Oligonucleotides and Hybridization

Using synthetic 3′-amine-modified oligonucleotides of 18 mer and 24 mer lengths, we tested the surface attachment capabilities. Each of these oligonucleotides had Watson-Crick complementary strands labeled with Cy5 dye. The 18 mer had a sequence: 5′-Cy3-ACCACCAAGCGAAACATC-C6-Amine-3′, with its a complementary oligonucleotide sequence having: 5′-Cy5-ATGTTTCGCTTGGTGGTC-3′. The 24 mer has a sequence: 5′-(Cy3)CACAGGGGAGGTGATAGCATTGCT(Amine)-3′, with its complementary oligonucleotide sequence for hybridization with: 5′-(Cy5)-AGCAATGCTATCACCTCCCCTGTG-3′. We applied gel filtration purification to remove any amine contamination.

A 10-50 μM concentration of the oligomers in 0.1M sodium borate buffer (pH 9.2) was recommended for either pin-printing (e.g., the Flexy robotic printer) or ink-jet printing. After the oligonucleotide solution was spotted or printed on the SMA activated slides to form an array, the slides were kept in a humidity chamber at room temperature for 1-4 hours to allow the reaction to go to completion.

Residual active anhydride groups were blocked using a 0.1M solution of ethanolamine in Tris buffer (0.1M, pH 9.0). After being pre-wanned to 50° C., the blocking solution was reacted with the slide surface for about 15 minutes at 50° C. Following the blocking step, a solution of 2×SSC/0.1% SDS was used to wash the slides. Once at 50° then three times at room temperature. The slides were then rinsed with de-ionized water three times and dried with a stream of clean nitrogen gas.

Hybridization was carried out in a hybridization chamber. A synthetic complementary oligomer labeled with Cy5 dye was used. The recommended hybridization solution was 5×SSC/0.1% SDS0.1% BSA at an appropriate temperature that is dependent on the probe size. After hybridization, the slide was washed with 5×SSC/0.1% SDS. Once at the hybridization temperature and twice at room temperature. The slide was washed three times with 2×SSC and three times with deionized water. After using a stream of clean nitrogen gas to dry the slide, the samples were scanned by using either a confocal or a CCD scanner.

C. Impact of pH and Concentration of Oligonucleotides on Attachment

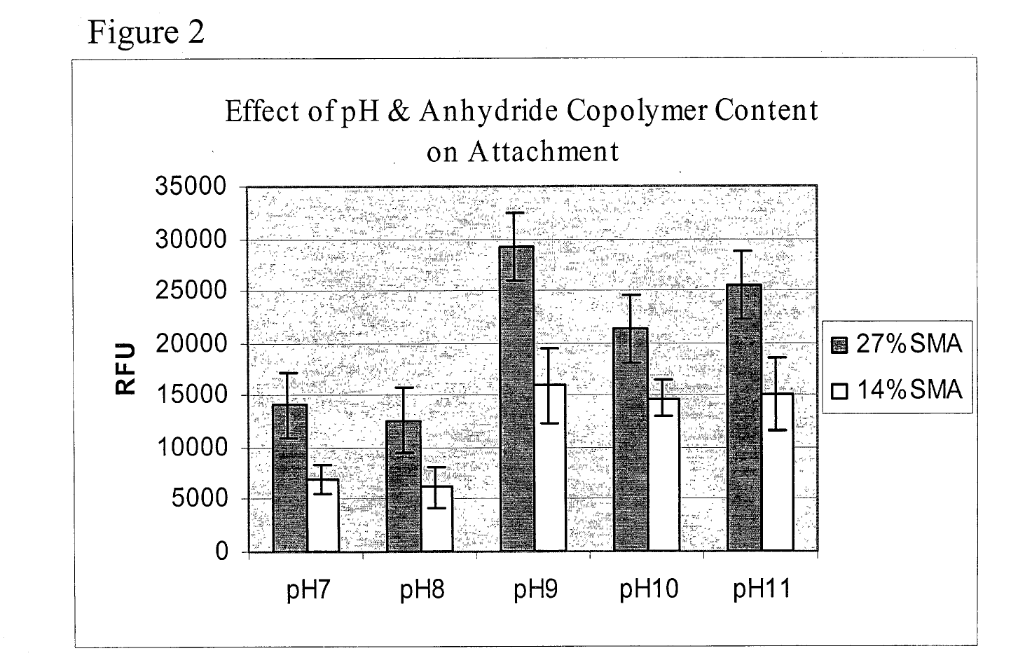

Using slides coated with 27% SMA and 14% SMA, we spotted about 0.25 μL of a solution of the amine-modified 24mer, each having 20 μM concentration, in five different buffers. The five buffers used were: 2×SSC (pH 7); HEPES (50 mM, pH 8); sodium borate (100 mM, pH 9.2); sodium bicarbonate (50 mM, pH 10); and sodium phosphate (100 mM, pH 11). After performing hybridization with Cy5-labeled complementary oligonucleotide, we observed that for both 27% and 14% SMA-coated substrates a higher pH level generally gives better oligonucleotide attachment efficiency to the surface. FIG. 2 shows the results. A pH level of about 9 is more preferred.

At six different concentrations (i.e., 1, 5, 10, 25, 50, 100 μM) of the amine-modified 24mer, prepared in 0.15 M sodium borate buffer, pH 9.2, we pin-printed oligonucleotides onto the 27% and 14% SMA-coated slides. After hybridization with Cy5-labeled complementary oligonucleotides, we observed a higher efficiency of oligonucleotide attachment to the coated surface. As shown in FIG. 2, the concentrations of oligonucleotide for immobilization work well at levels greater than about 15 μM. Preferred concentrations are about 20-100 μM, or more.

D. Impact of Anhydride Content on Oligonucleotide Attachment

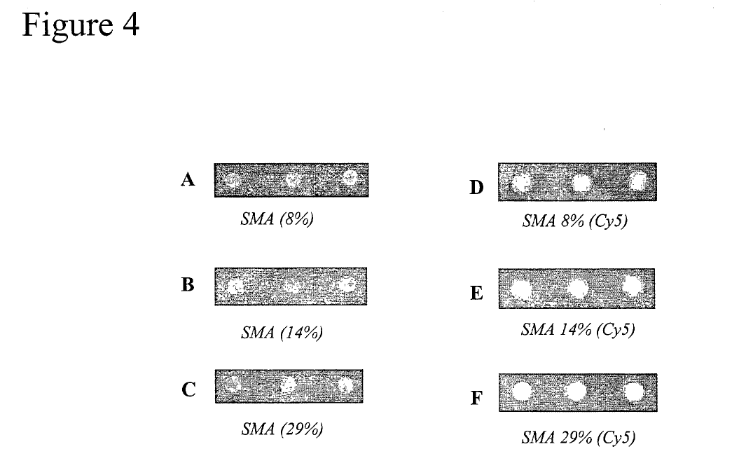

We applied a polymer coating with an anhydride-copolymer content ranging from 8%, 14%, and 29%, respectively, on to three Corning GAPS-coated slides. To avoid variations due to delivery by contact pin-printing and differences in surface properties we used a piezo-electric printer to deposited three duplicate spots of Cy3-labeled, aminated 18 mer oligonucleotides on each of the slides, under the same delivery condition in borate buffer (pH 9.2/20% DMF). After hybridizing with Cy5-labeled complementary 18 mer oligonucleotides, the resulting data, summarized in FIGS. 4A-4F, indicated that a higher anhydride content improves both oligonucleotide immobilization and hybridization.

### Example 2

Alternate Preparation of Maleic Anhydride Presenting Substrates

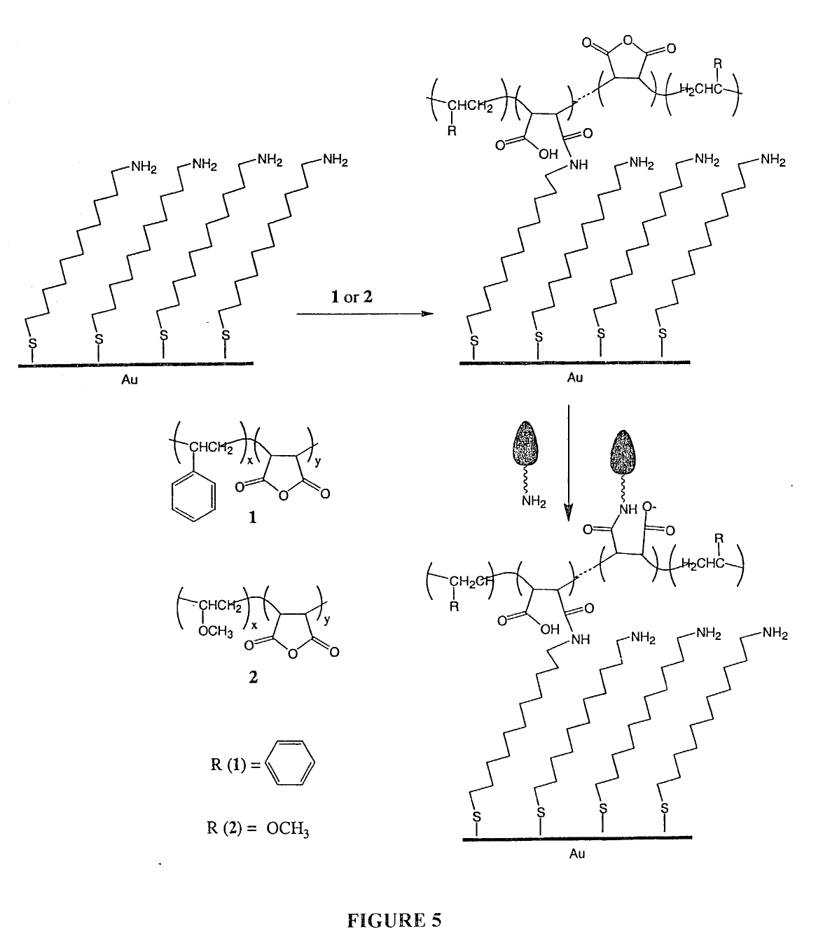

Preparation of Surfaces: As shown in FIG. 5, to produce self-assembled monolayers (SAMs), gold-coated substrates were soaked for 1-2 hours in ethanolic solutions (1 mM or 2 mM) of 11-mercaptoundecylamine. These substrates were then rinsed with ethanol and dried. The conjugation of polymers to the substrate was accomplished by immersion in solutions of the polymer in DMSO (10 mg/mL) containing ˜0.1% triethylamine for 1 hr. The substrates were then rinsed with DMSO, ethanol, and dried.

Alternatively, polymers can be coupled to the surface by immersing the substrate for 1 hour in a 10 mg/mL solution of the polymer in methyl-ethyl-ketone containing 0.1% triethylamine. The substrates are then rinsed with ethanol and distilled water and dried. (The polymer poly(maleic anhydride-alt-methyl vinyl ether) is commercially available from Aldrich; poly(tri(ethylene glycol methyl vinyl ether)-altmaleic anhydride) was synthesized in-house via free radical polymerization.) The substrates are rinsed by soaking for 10 minutes in pure methyl ethyl ketone followed by an ethanol and drying with nitrogen.

Using ellipsometry, we characterized the attachment of poly(maleic anhdyride-alt-methyl-vinyl ether) (“MAMVE”, structure 2 in FIG. 5) to amine-presenting SAMs, and the subsequent attachment of amine-containing molecules to the reactive surface. Table 1 summarizes the increases in thickness of SAMs presenting different functional groups after being reacted with MAMVE. Among the surfaces tested, only SAMs presenting amine groups showed an increase in thickness. If the polymer is immobilized with the polymer backbone parallel to the surface, the expected increase in thickness is ˜6-7 Å, which corresponds to the observed increase in thickness. We hypothesize that a monolayer of the polymer is conjugated to the SAM to form a comb-like structure.

TABLE 1
Ellipsometric increases in thickness (Δd) after reaction with methyl-vinyl-
ether-co-maleic anhydride polymer (MAMVE), and after subsequent
reaction of the anhydride-presenting surface with undecylamine (UA)

| SAM | Δd (+ (MAMVE)) (Å) | Δd (+ (UA)) (Å) |
|---|---|---|
| HSC₁₁NH₂ | 7.1 ± 1.1^{a} | 5.2 ± 0.8^{b} |
| HSC₁₆ | 0 | — |
| HSC₁₀COOH | 0 | — |
| HSC₁₁OH | 0 | — |

^{a}average of 8 samples; ^{b}n-average of 3 samples

To ascertain the amount of coupling to the polymer (anhydride)-modified surface, the substrate was immersed in a solution of undecylamine (UA)(10 mM) in DMSO for 1.5 hours. After derivatization with undecylamine, the thickness of the surface increased by ˜5 Å (Table 1). A packed monolayer of undecylamine would give an ellipsometric thickness of ˜17 Å; thus, the observed increase in thickness corresponds to approximately ˜30% coverage of the surface.

To determine whether the attachment of MAMVE to the amine-SAM was covalent or electrostatic, we determined whether the observed increase in thickness was reversible or not. An irreversible increase in thickness would suggest covalent attachment; conversely, a reversible increase in thickness would suggest non-covalent attachment. We found that there was no decrease in the thickness of the substrate after washing with acidic buffer (pH˜3). In another experiment, MAMVE was hydrolyzed by stirring overnight in a solution of ammonia. The adsorption of this hydrolized polymer to the amine-presenting SAM resulted in an increase in thickness corresponding to ˜8.6 Å; this adsorption is probably due to electrostatic interactions between the negatively charged polymer and the positively charged surface. There was, however, no subsequent increase in thickness after reaction with undecylamine. Moreover, soaking the surface in an acidic buffer (pH3) resulted in a large decrease in the thickness. At this pH, the carboxylate groups of the hydrolyzed polymer get protonated to form carboxyl groups, which would greatly decrease the affinity of the polymer for the surface and lead to its desorption.

Protein Binding to Substrates: Gold-coated substrates obtained from BIAcore, derivatized as described above, were incorporated into the BIAcore cassettes using the sensor-chip assembly unit supplied by the manufacturer. The cassettes were docked into the BIAcore 2000 SPR instrument and the surfaces was equilibrated with buffer solution (HEPES, 10 mM, pH 7.4 containing 150 mM NaCl, 3 mM EDTA, and ˜0.005% or 0.006% TWEEN 20). Solutions of protein (0.5 mg mL⁻¹, in pH 8 borate buffer) was injected over the surface for 20 min to react with residual maleic anhydride groups. The system was then returned to buffer and the substrates were readied for protein-binding studies.

For IgG/anti-IgG experiments, a solution of 0.5 mg/mL human IgG in borate buffer (200 mM, pH 8.5) was injected over the surface for 7 minutes. The system was then returned to buffer for 2 minutes and the surface was blocked by either i) a 7 minute injection of ethanolamine (500 mM in borate buffer, pH 8.5); ii) a 2 minute injection of DEAE dextran (0.1 mg/mL in borate buffer, pH 8.5). A 0.1 mg/mL solution of anti-human IgG in phosphate buffered saline was injected over the surface for 7 minutes.

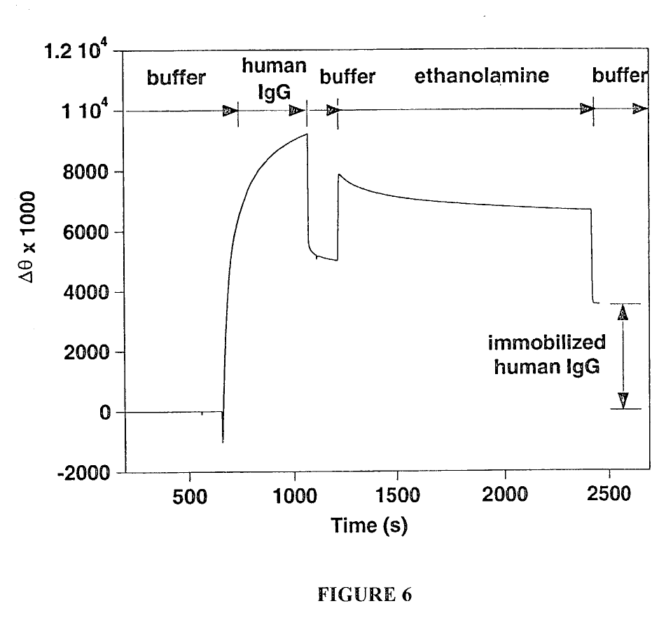

A sensorgram corresponding to the immobilization of human IgG is shown in FIG. 6. The immobilization of the antibody results in a changes the SPR angle (Δθ) by ˜0.35°, which corresponds to ˜3.5 ng mm⁻² of adsorbed protein.

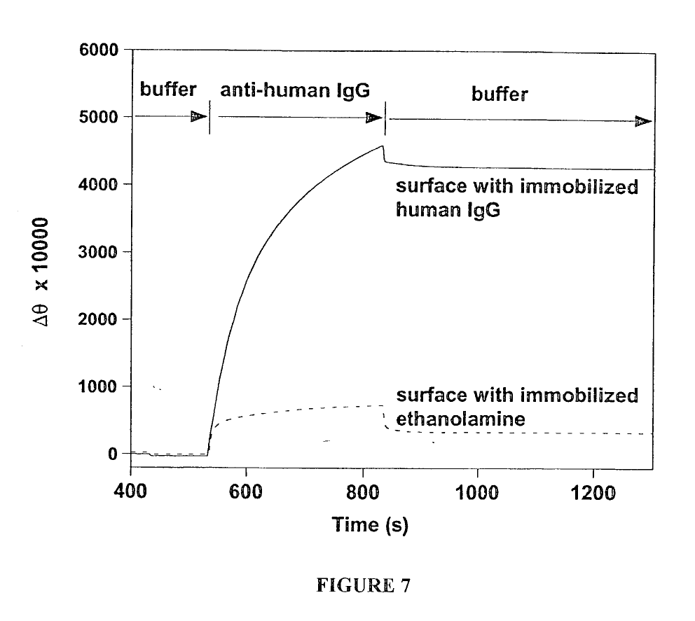

Blinding of Proteins to Immobilized Proteins and Ligands. Binding studies were conducted inside the SPR machine. Solutions of goat anti-human IgG (0.1 mg mL⁻¹) were injected over surfaces with immobilized human IgG or ethanolamine. FIG. 7 shows that the amount of binding of the anti-human IgG antibody on surfaces presenting human IgG (Δθ ˜0.42°) derivatized with ethanolamine (Δθ ˜0.030°); we infer that the binding is specific. These data suggest the following: (i) the lack of protein binding to the ethanolamine derivatized surface implies that immobilization of protein occurs on anhydride presenting surfaces and does not occur on deactivated surfaces; and (ii) proteins immobilized on the anhydride surfaces can be used for studies of biospecific binding.

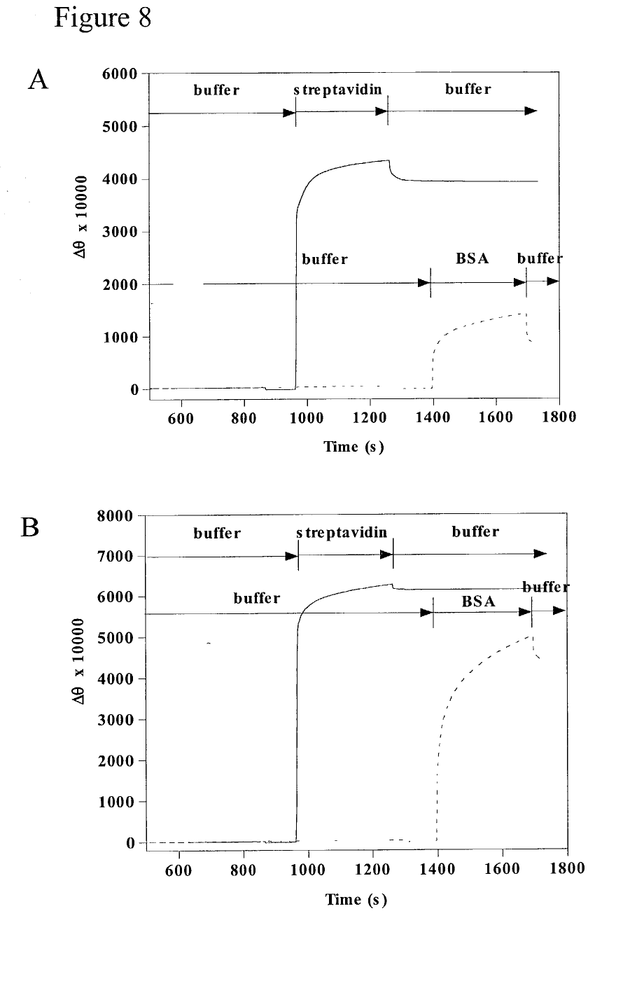

We also compared the binding of proteins to ligands immobilized on MAMVE, with binding of proteins to ligands immobilized on styrene-maleic anhydride (structure 1, FIG. 5). Biotin was immobilized by injecting solutions of 5-(biotinamido)-pentylamine over surfaces presenting polymer 1 or 2. Solutions of streptavidin (1 μM) or BSA (as a control to test specificity) were injected over these surfaces. FIG. 8A shows the amounts of binding of streptavidin and BSA to biotin groups immobilized on MAMVE. FIG. 11B shows the corresponding data for a poly(styrene maleic anhydride) presenting surface. Data indicate that the amount of non-specific binding of proteins on surfaces presenting styrene side chains is considerably greater than that on surfaces presenting methyl ethers. Non-specific binding of proteins to surfaces such as those presenting hydrophobic aromatic groups is well documented; the inertness of surfaces presenting —OCH₃ groups to non-specific adsorption has also been observed (Chapman, R. G. et al., J. Am. Chem. Soc. 2000, 122, 8303-8304).

### Example 3

Electrostatic Blocking of Surfaces Modified with Anhydride-Containing Polymers

According to the invention, we employ electrostatic blocking agents on anhydride-modified surfaces. Diethylaminethyl (DEAE) dextran is particularly effective in reducing the non-specific binding of proteins to surfaces modified with poly(maleic anhydride-alt-methyl vinyl ether) or SMA.

To demonstrate the use of DEAE dextran as an electrostatic blocking agent, chemically modified gold surfaces were prepared containing a thin (˜1.5 nm) layer of poly(maleic anhydride-alt-methyl vinyl ether) attached to a self-assembled monolayer of 11-mercaptoundecylamine (MUAM). After being docked into the Biacore 2000 surface plasmon resonance (SPR) instrument and equilibrated with buffer, these surfaces were reacted with ethanolamine, and then blocked for 2 minutes with either i) ethanolamine; ii) DEAE dextran, a positively charged dextran; iii) carboxymethyl dextran, a negatively charged dextran; or iv) native dextran, which is uncharged. The amount of protein which bound to each surface was determined by injecting a solution of protein (0.5 mg/mL each of fibrinogen, lysozyme, concanavalin A, and bovine serum albumin in phosphate buffered saline, pH 7.4) over the surface for 7 minutes. (For the Biacore instrument, 1000 RU corresponds to ˜1 ng/mm² of adsorbed protein). Following this injection, the system was returned to buffer and washed for 2-20 minutes. FIG. 9 summarizes the results of this experiment. Notice that the surface blocked with ethanolamine binds a significant amount of protein. In contrast, the surface blocked with DEAE-dextran shows substantially less binding. Specifically, after a 2-minute buffer wash, the surface blocked with ethanolamine bound ˜3.1 ng/mm² (3100 RU) of protein whereas the DEAE-dextran blocked surface bound only 0.74 (740 RU) of protein. Similar amounts of protein were observed to bind to surfaces blocked with either carboxymethyl dextran or native dextran, suggesting that these dextrans do not bind to the surface and that the interaction between the polymer surface and DEAE dextran is electrostatic.

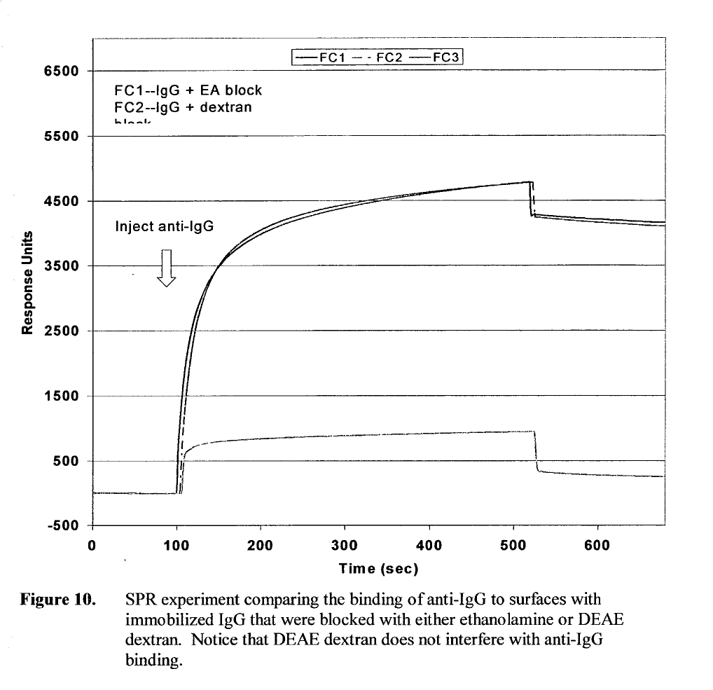

One concern with the use of a polymeric blocking agent such as DEAE-dextran is the possibility that it might interfere with the ability of analytes to bind to immobilized targets. To address this question, an SPR experiment was performed in which human IgG was immobilized on a poly(tri(ethylene glycol methyl vinyl ether)-alt-maleic anhydride) modified gold surface. Following this immobilization, flow channel 1 (FC1) was blocked with EA and flow channel 2 (FC2) was blocked with EA+DEAE dextran. Both channels were then injected with a solution of anti-IgG. As can be seen in FIG. 10, similar amounts of anti-IgG bound to both channels indicating that DEAE dextran does not interfere with IgG/anti-IgG binding.

Although the present invention has been described generally and in detail by way of examples, persons skilled in the art will understand that the invention is not limited necessarily to the embodiments specifically disclosed, but that modifications and variations can be made without departing from the spirit and scope of the invention. Therefore, unless changes otherwise depart from the scope of the invention as defined by the following claims, they should be construed as included herein.

We claim:

1. A substrate for supporting a biological array, the substrate comprising:
a reactive surface to which a polymer coating can attach by covalent bonds;
an even coating of a polymer containing functional groups, which can reduce nonspecific binding of various biomolecules to a polymer-coated substrate surface.

2. The substrate according to claim 1, wherein said biomolecules attach to said polymer-coated substrate in sufficient amounts under about 6 hours.

3. The substrate according to claim 2, wherein said biomolecules attach to said polymer-coated substrate in sufficient amounts within about 5.5 hours.

4. The substrate according to claim 1, further comprising an intermediate tie-layer on at least a surface of said substrate to enhance covalent bonds between said substrate and said polymer coating.

5. The substrate according to claim 4, wherein the tie-layer comprises reactive polar moieties.

6. The substrate according to claim 5, wherein said reactive polar moieties may include: amino group, thiol group, hydroxyl group, carboxyl group, acrylic acid, other organic and inorganic acid, esters, anhydrides, aldehydes, epoxides, and their derivatives or salts.

7. The substrate according to claim 5, wherein said reactive polar moieties moieties may be straight or branched-chain aminosilane, aminoalkoxysilane, aminoalkylsilane, aminoarylsilane, aminoaryloxysilane, derivatives or salts thereof.

8. The substrate according to claim 7, wherein said aminoalkylsilane moieties may include: γ-aminopropyl trimethoxysilane, N-(beta-aminoethyl)-γ-aminopropyl trimethoxysilane, N-(beta-aminoethyl)-γ-aminopropyl triethoxysilane or N′-(beta-aminoethyl)-γ-aminopropyl methoxysilane.

9. The substrate according to claim 4, wherein the tie-layer is attached to the substrate by covalent binding or other strong chemical interactions.

10. The substrate according to claim 4, wherein the tie-layer comprises a self-assembled monolayer (SAM).

11. The substrate according to claim 10, wherein the SAM comprises 11-mercaptoundecylamine or other amine-terminated alkanethiols.

12. The substrate according to claim 1, wherein the substrate includes any stable solid of a desired dimension selected from either a plastic, a polymer or co-polymer substance, a ceramic, a glass, a metal, a crystalline material, or any combinations thereof, or a coating of one material on another.

13. The substrate according to claim 12, wherein the substrate is of a (semi) noble metal; glass material; metallic or non-metallic oxides; crystalline material; transition metal; and plastic, polymers or copolymers.

14. The substrate according to claim 1, wherein the substrate is a planar slide made from a borosilicate or boroaluminosilicate glass.

15. The substrate according to claim 1, wherein the polymer is either linear or non-linear

16. The substrate of according to claim 1, wherein the polymer coating comprises a copolymer.

17. The substrate according to claim 16, wherein the copolymer may comprise both hydrophilic and hydrophobic units.

18. The substrate according to claim 1, wherein the polymer coating comprises an anhydride functional group.

19. The substrate according to claim 18, wherein the polymer coating comprises a maleic anhydride and another copolymer unit.

20. The substrate according to claim 17, wherein said copolymer comprises: maleic anhydride, styrene, tetradecene, octadecene, methyl vinyl ether, triethylene glycol methyl vinyl ether, butylvinyl ether; or divinylbenzene.

21. The substrate according to claim 16, wherein the polymer or copolymer may include: poly(divinylbenzene), poly(methyl methacrylate), poly(vinyl acetate-maleic anhydride), poly(dimethylsiloxane) monomethacrylate; copolymers such as poly(styrene-co-maleic anhydride), poly(styrene-co-butadiene), poly(styrene-co-divinylbenzene), poly(ethylene-alt-maleic anhydride), poly(isobutylene-alt-maleic anhydride), poly(maleic anhydride-alt-1-octadecene), poly(maleic anhydride-alt-1-tetradecene), poly(2-vinylpyridine-co-styrene), poly(styrene-co-vinylbenzyl chloride-co-divinylbenzene), poly(styrene-co-vinylbenzylamine-co-divinylbenzene), poly(maleic anhydride-alt-methyl vinyl ether).

22. The substrate according to claim 1, wherein the polymer coating is at least a monolayer.

23. The substrate according to claim 1, wherein the polymer coating has a thickness of about 20 Å-1000 Å.

24. The substrate according to claim 1, the polymer coating has a thickness of up to a few centimeters.

25. The substrate according to claim 1, wherein said biomolecules exhibit specific affinity for another molecule through covalent or non-covalent bonding.

26. The substrate according to claim 1, wherein said biomolecules include: natural or synthetic oligonucleotides; natural or modified/blocked nucleotides/nucleosides; nucleic acids (DNA) or (RNA); proteins or fragments of proteins; peptides which may contain natural or modified/blocked amino acids; antibodies; haptens; biological ligands; protein membranes; lipid membranes; and cells.

27. The substrate according to claim 1, wherein said biomolecules are oligonucleotides.

28. The substrate according to claim 27, wherein said oligonucleotides are from about 5 to about 500 nucleotides.

29. The substrate according to claim 28, wherein said oligonucleotides are from about 5 to about 200 nucleotides.

30. The substrate according to claim 29, wherein said oligonucleotides are from about 10 to about 100 nucleotides.

31. The substrate according to claim 1, further comprising a charged compound that has good non-specific binding properties itself, when binding proteins.

32. The substrate according to claim 31, wherein said charged compound is positively charged.

33. The substrate according to claim 31, wherein said compound includes a positively charged dextran to negate a negatively charged surface of the substrate for binding proteins.

34. A method for preparing a substrate to support an array of biomolecules, the method comprising: providing a substrate of a suitable material; preparing on the substrate a reactive surface for attaching a polymer coating; and, applying an polymer coating in an even layer to the reactive surface of the substrate.

35. The method according to claim 34, wherein said polymer-coated substrate can attach biomolecules in sufficient amounts to form microspots within about 5.5 hours.

36. The method according to claim 34, wherein said preparing step may further comprise forming an intermediate tie-layer on at least a surface of said substrate to enhance covalent bonds between said substrate and said polymer coating.

37. The method according to claim 36, wherein the tie-layer comprises reactive polar moieties.

38. The method according to claim 37, wherein said reactive polar moieties may include: amino group, thiol group, hydroxyl group, carboxyl group, acrylic acid, other organic and inorganic acid, esters, anhydrides, aldehydes, epoxides, and their derivatives or salts.

39. The method according to claim 37, wherein said reactive polar moieties moieties may be straight or branched-chain aminosilane, aminoalkoxysilane, aminoalkylsilane, aminoarylsilane, aminoaryloxysilane, derivatives or salts thereof.

40. The method according to claim 39, wherein said aminoalkylsilane moieties may include: γ-aminopropyl trimethoxysilane, N-(beta-aminoethyl)-γ-aminopropyl trimethoxysilane, N-(beta-aminoethyl)-γ-aminopropyl triethoxysilane or N′-(beta-aminoethyl)-γ-aminopropyl methoxysilane.

41. The method according to claim 36, wherein the tie-layer is attached to the substrate by covalent binding or other strong chemical interactions.

42. The method according to claim 36, wherein the tie-layer comprises a self-assembled monolayer (SAM).

43. The method according to claim 42, wherein the SAM comprises 11-mercaptoundecylamine or other amine-terminated alkanethiols.

44. The method according to claim 34, wherein when binding proteins, further comprising applying a charged compound that has good non-specific binding properties itself, after attachment of a biomolecule to said surface but before a detection step.

45. The method according to claim 44, wherein said charged compound negates a substrate surface of an opposite charge.

46. The method according to claim 44, wherein said compound includes a positively charged dextran to negate a negatively charged surface of the substrate for binding proteins.

47. The method according to claim 34, wherein said polymer coating comprises functional groups, which can reduce nonspecific binding of various biomolecules to a polymer-coated substrate surface, can attach by covalent bonds to said reactive substrate surface, and can immobilize various biological molecules to said polymer-coating.

48. The method according to claim 34, wherein the polymer is either linear or non-linear

49. The method of according to claim 34, wherein the polymer coating comprises a copolymer.

50. The method according to claim 49, wherein the copolymer may comprise both hydrophilic and hydrophobic units.

51. The method according to claim 34, wherein the polymer coating comprises an anhydride functional group.

52. The method according to claim 51, wherein the polymer coating comprises a maleic anhydride and another copolymer unit.

53. The method according to claim 51, wherein said copolymer comprises: maleic anhydride and styrene, tetradecene, octadecene, methyl vinyl ether, triethylene glycol methyl vinyl ether, butylvinyl ether; or divinylbenzene.

54. The method according to claim 34, wherein the polymer or copolymer may include: poly(divinylbenzene), poly(methyl methacrylate), poly(vinyl acetate-maleic anhydride), poly(dimethylsiloxane) monomethacrylate; copolymers such as poly(styrene-co-maleic anhydride), poly(styrene-co-butadiene), poly(styrene-co-divinylbenzene), poly(ethylene-alt-maleic anhydride), poly(isobutylene-alt-maleic anhydride), poly(maleic anhydride-alt-1-octadecene), poly(maleic anhydride-alt-1-tetradecene), poly(2-vinylpyridine-co-styrene), poly(styrene-co-vinylbenzyl chloride-co-divinylbenzene), poly(styrene-co-vinylbenzylamine-co-divinylbenzene), poly(maleic anhydride-alt-methyl vinyl ether).

55. The method according to claim 34, wherein the polymer coating is at least a monolayer.

56. The method according to claim 34, wherein the polymer coating has a thickness of about 20 Å-1000 Å.

57. The method according to claim 34, the polymer coating has a thickness of up to a few centimeters.

58. The method according to claim 34, wherein the substrate includes any stable solid of a desired dimension selected from either a glass, a ceramic, a metal, a crystalline material, a plastic, a polymer or co-polymer substance, or any combinations thereof, or a coating of one material on another.

59. The method according to claim 34, wherein the substrate is of a (semi) noble metal; glass material; metallic or non-metallic oxides; crystalline material; transition metal; and plastic, polymers or copolymers.

60. The method according to claim 34, wherein the substrate is a planar slide made from a borosilicate or boroaluminosilicate glass.

61. The method according to claim 34, wherein said biomolecules exhibit specific affinity for another molecule through covalent or non-covalent bonding.

62. A method for making a biological array, the method comprising: providing a substrate; preparing a reactive surface on said substrate for attaching a polymer coating; applying a polymer coating to the reactive surface of the substrate; and, depositing biomolecules onto said polymer-coated surface.

63. The method according to claim 62, wherein said biomolecules attach to said polymer-coated substrate in sufficient amounts under about 6 hours.

64. The method according to claim 62, wherein said biomolecules attach to said polymer-coated substrate in sufficient amounts within about 5.5 hours.

65. The method according to claim 62, wherein said preparing step may further comprise forming an intermediate tie-layer on at least a surface of said substrate to enhance covalent bonds between said substrate and said polymer coating.

66. The method according to claim 65, wherein the tie-layer comprises reactive polar moieties.

67. The method according to claim 65, wherein said reactive polar moieties may include: amino group, thiol group, hydroxyl group, carboxyl group, acrylic acid, other organic and inorganic acid, esters, anhydrides, aldehydes, epoxides, and their derivatives or salts.

68. The method according to claim 65, wherein said reactive polar moieties moieties may be straight or branched-chain aminosilane, aminoalkoxysilane, aminoalkylsilane, aminoarylsilane, aminoaryloxysilane, derivatives or salts thereof.

69. The method according to claim 68, wherein said aminoalkylsilane moieties may include: γ-aminopropyl trimethoxysilane, N-(beta-aminoethyl)-γ-aminopropyl trimethoxysilane, N-(beta-aminoethyl)-γ-aminopropyl triethoxysilane or N′-(beta-aminoethyl)-γ-aminopropyl methoxysilane.

70. The method according to claim 65, wherein the tie-layer is attached to the substrate by covalent binding or other strong chemical interactions.

71. The method according to claim 65, wherein the tie-layer comprises a self-assembled monolayer (SAM).

72. The method according to claim 71, wherein the SAM comprises 11-mercaptoundecylamine or other amine-terminated alkanethiols.

73. The method according to claim 62, further comprising treating said polymer-coated surface with other chemical reagents to create a stable attachment having reduced background signal.

74. The method according to claim 62, wherein when binding proteins, further comprising applying a charged compound that has good non-specific binding properties itself, after attachment of a biomolecule to said surface but before a detection step.

75. The method according to claim 74, wherein said compound includes a positively charged dextran to negate a negatively charged surface of the substrate for binding proteins.

76. The method according to claim 62, wherein said polymer coating comprises functional groups, which can reduce nonspecific binding of various biomolecules to a polymer-coated substrate surface, can attach by covalent bonds to said reactive substrate surface, and can immobilize various biological molecules to said polymer-coating.

77. The method according to claim 62, wherein the polymer is either linear or non-linear

78. The method of according to claim 62, wherein the polymer coating comprises a copolymer.

79. The method according to claim 78, wherein the copolymer may comprise both hydrophilic and hydrophobic units.

80. The method according to claim 62, wherein the polymer coating comprises an anhydride functional group.

81. The method according to claim 80, wherein the polymer coating comprises a maleic anhydride and another copolymer unit.

82. The method according to claim 81, wherein said copolymer comprises: maleic anhydride and styrene, tetradecene, octadecene, methyl vinyl ether, triethylene glycol methyl vinyl ether, butylvinyl ether; or divinylbenzene.

83. The method according to claim 62, wherein the polymer or copolymer may include: poly(divinylbenzene), poly(methyl methacrylate), poly(vinyl acetate-maleic anhydride), poly(dimethylsiloxane) monomethacrylate; copolymers such as poly(styrene-co-maleic anhydride), poly(styrene-co-butadiene), poly(styrene-co-divinylbenzene), poly(ethylene-alt-maleic anhydride), poly(isobutylene-alt-maleic anhydride), poly(maleic anhydride-alt-1-octadecene), poly(maleic anhydride-alt-1-tetradecene), poly(2-vinylpyridine-co-styrene), poly(styrene-co-vinylbenzyl chloride-co-divinylbenzene), poly(styrene-co-vinylbenzylamine-co-divinylbenzene), poly(maleic anhydride-alt-methyl vinyl ether).

84. The method according to claim 62, wherein the polymer coating is at least a monolayer.

85. The method according to claim 62, wherein the polymer coating has a thickness of about 20 Å-1000 Å.

86. The method according to claim 62, the polymer coating has a thickness of up to a few centimeters.

87. The method according to claim 62, wherein the substrate includes any stable solid of a desired dimension selected from either a glass, a ceramic, a metal, a crystalline material, a plastic, a polymer or co-polymer substance, or any combinations thereof, or a coating of one material on another.

88. The method according to claim 62, wherein the substrate comprises a (semi) noble metal selected from gold, silver, and platinum; glass material; metallic or non-metallic oxides; crystalline material; transition metal; and plastic, polymers or copolymers.

89. The method according to claim 62, wherein the substrate is a planar slide made from a borosilicate or boroaluminosilicate glass.

90. The method according to claim 62, wherein said biomolecules include: natural or synthetic oligonucleotides; natural or modified/blocked nucleotides/nucleosides; nucleic acids (DNA) or (RNA); proteins or fragments of proteins; peptides which may contain natural or modified/blocked amino acids; antibodies; haptens; biological ligands; protein membranes; lipid membranes; and cells.

91. The method according to claim 62, wherein said biomolecules are oligonucleotides.

92. The method according to claim 91, wherein said oligonucleotides are from about 5 to about 500 nucleotides.

93. The method according to claim 91, wherein said oligonucleotides are from about 5 to about 200 nucleotides.

94. The method according to claim 91, wherein said oligonucleotides are from about 10 to about 100 nucleotides.

95. A method for reducing non-specific binding of proteins to a surface of a biological array device, the method comprising: contacting said surface with a charged compound that has good non-specific binding properties itself, after attachment of a biomolecule to said surface, but before a detection step.

96. The method of claim 95, wherein said charged compound negates a substrate surface of an opposite charge.

97. The method of claim 95, wherein the surface of said biological array device comprises an anhydride-containing polymer.

98. The method of claim 95, wherein the charged compound includes a positively charged dextran.

99. The method of claim 95, wherein said charged compound is diethylaminethyl (DEAE) dextran.

100. The method of claim 95, wherein said biological array device includes any stable solid of a desired dimension selected from either a glass, a ceramic, a metal, a crystalline material, a plastic, a polymer or co-polymer substance, or any combinations thereof, or a coating of one material on another.
